SEQUENCE LISTING (9 sequences)

SEQ ID NO: 1 - DNA, 4191 bp, Gasterosteus aculeatus
catcaggcagcatgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgtccaggtccacacgaccattggtataaacttccaaattcagatgtcccctgaaatccatctgttcatcgacgcacctgacccctccagtgacaagattaaaggtctttgtggcaatagcaacagtgacaccacagatgacttcaccaccaacagcgggatcattgagaactcagctaaaccatttgctatgtcctggagtttgcttaattgtttcggtaacatacccaccacctgcaccaacctggagaatgagaattatgctcatgaaaagtgtgcagtgttaaaccaaccaactgggatatttgctaagtgccaccctcatatcccaactgattactactacacggcttgcatccaaagaatatgtaactctgccggaagtcggagacagggcttgtgtattggtctggccagctacgccaaagcctgcgccggtgttggtgttgtaattggtgactggaggagaatcacgggctgcgatctgaaatgccagaagaaccaagaattctcctacagcatgcatacatgcaaccgcacatgcaattctctgaccggccatgacatccgctgtggtatgaatgacgatgctgtggagggctgtggctgtccggagggaactcacctgaaccaaggacagacctgttgcccaaaggaagagtgtggttgtatttactacggtggtattgcagccccggggcctgttgttatcgccggacaaaagtgcgactgcaagaatgggatactgaactgcttgccgaattgtgattgcagaaatgggaaggtgtgtgtcagttgctctgagggccaacataagagggttcagaagacctgtgactatattagcaaaccaaagggtaccagggagaactgtaagagtggctgttactgtccagatcaccagtatgaagatcaccatgggaactgtgtttcactcgatgattgcacctgtgtgttcagtggcaaagcattcaaagctggacagcaagttaccagcaactgtaaaacatgtacctgttatcgtggtcagtggcactgcattgagaagccctgcccgggacagtgccaagtctacggaaatggacactaccagacctttgactccaaatggttccgcttttctggacaatgtctgtacacacttgtgcaggattcctgtgacatgagaagaggcaccttctctatcagagtggagagtgtcccctgctgtgaggaggtgctcacctgctctcgcaacatcatccttgacctgaagggccaagtcaccctgacgctgagagacatgcaggtgaccagacgcctccatgaaggctggactgggcaggatgattcactttactcaatacacactttgggactttaccaacatgcgctctgacgacagagtcaacaattcaatgcccagcatggccacgacagcagtgggcgtgtccatcttcctgctgaccaccaccgcctgcctggtggtcaagtcccgccttatcccgtgtcaatcacacagccggcgctcttcagaccagctggacctgatggtggacggacttcccgtctccctcccctcctacgaggaggcgatgtacagcagctgggggcagcgcctccccgccttctcagcgccggggggccccacccagctcctactggctcaagaggctcccagctgtcacccagcggctctcaacaatcaagacagcagcaaccgcccccccctgcccagccccgacaacccgccgcccccgtacgaggaggtgcagtcgtcacaacatagggacaggatgggggacggggacatccggccgctccgcgtcgcgttttcagatgacaaggacacttgattgacagactgcagggtttacatgtgattacgtgtttagcaatgaaggattttgattgacagctgcggtgtaaaaatacatccagtactgcactttttctgactggctgcagctcaccagcatcacatgctaatttagaaaaagctgataagctctgattggccggcagtgaggtcacaaagacctgaaccatttctacgggaggtattgattgtaaagtgtgtctattgaatgtgagacaagggcacttgtgctttatgccgaaacctttagctcccgaatgccttatttttagtgtgatgacagtacgttttattggaattatgagtgtgtttctggagtcggtgagagtgtggattgtgctgttgcagtacaaaaatcctgtgcgtttccacggcaactgactccatctctatctgaatctacctttggtaaagtgtttctcttggtggtgttgatttaaagccagcgtggtgcacagtgaaggtactaattacatgctcgtgtgcggacactaggacgatagattgtgacaaacaagcaccattttgtgatatttattttgtgtatatcaaatatttagagaagttacaatgatcggatgtgttcatttactggtgagtgaaggaaaattttatattttgattaattgaaatgtctaatcaagagaatataacattattgtgcatttgctccatcagtggagactcacaaagacgacagttttcattctggaccaaataaccccaaagtgcgtgcgtgcgtgcgtgcgcatgcatcttcacttcaccaggctacttccccctctagcctctgcagagcgaccaacgcctttacagaccttgaatgtattttatgtttcatattgtggcaacaaatcctaactggattgtaatatttaccaaaagattatttgtacatttgtgttattgcacagaattatatgatcaataaatgttgacgtttgaaaaaaaaaaaaaaaaaaaaaaa

SEQ ID NO: 2 - DNA, 2733 bp, Gasterosteus aculeatus
atgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgtccaggtccacacgaccattggtataaacttccaaattcagatgtcccctgaaatccatctgttcatcgacgcacctgacccctccagtgacaagattaaaggtctttgtggcaatagcaacagtgacaccacagatgacttcaccaccaacagcgggatcattgagaactcagctaaaccatttgctatgtcctggagtttgcttaattgtttcggtaacatacccaccacctgcaccaacctggagaatgagaattatgctcatgaaaagtgtgcagtgttaaaccaaccaactgggatatttgctaagtgccaccctcatatcccaactgattactactacacggcttgcatccaaagaatatgtaactctgccggaagtcggagacagggcttgtgtattggtctggccagctacgccaaagcctgcgccggtgttggtgttgtaattggtgactggaggagaatcacgggctgcgatctgaaatgccagaagaaccaagaattctcctacagcatgcatacatgcaaccgcacatgcaattctctgaccggccatgacatccgctgtggtatgaatgacgatgctgtggagggctgtggctgtccggagggaactcacctgaaccaaggacagacctgttgcccaaaggaagagtgtggttgtatttactacggtggtattgcagccccggggcctgttgttatcgccggacaaaagtgcgactgcaagaatgggatactgaactgcttgccgaattgtgattgcagaaatgggaaggtgtgtgtcagttgctctgagggccaacataagagggttcagaagacctgtgactatattagcaaaccaaagggtaccagggagaactgtaagagtggctgttactgtccagatcaccagtatgaagatcaccatgggaactgtgtttcactcgatgattgcacctgtgtgttcagtggcaaagcattcaaagctggacagcaagttaccagcaactgtaaaacatgtacctgttatcgtggtcagtggcactgcattgagaagccctgcccgggacagtgccaagtctacggaaatggacactaccagacctttgactccaaatggttccgcttttctggacaatgtctgtacacacttgtgcaggattcctgtgacatgagaagaggcaccttctctatcagagtggagagtgtcccctgctgtgaggaggtgctcacctgctctcgcaacatcatccttgacctgaagggccaagtcaccctgacgctgagagacatgcaggtgaccagacgcctccatgaaggctggactgggcaggatgattcactttactcaatacacactttgggactttaccaacatgcgctctga

SEQ ID NO: 3 - protein, 910 aa, Gasterosteus aculeatus
MTTQRWILAFCLSLASVFGTVELFKTKEIQTYTCRTFGSGIVQPFKGESYYVRSDCPFKLTSFNVNRGEYSVTIRRGHNGLLVQVEIIINKVTTLLQNGHILVQNKSVSLPYDHTYQHIFKYGIYTRLRSSLLPFTVTWHNVHGGINSLWVTLESELCTDMCGLCGKQNVAGHRDELIRDSKLHDHRCKIRDPVLRKNHICRRFFLKTKNCLQDNNSHYHRLCKENICGFENSQSIFCPFFQEVASQCNQSRINRFWRRLTKCAKPRCPGDLIYEKKGPAFIPSCSNPNPAPFYQELTETCSCPEGKVLNNCEKGYRCIPKSSCSCEFAGKTYGNGEIRSSRCQSCTCDGGKWRCSENFCHRRCVIEGQFVTTFDGKQYVLPNKCLYVASKGPNWIIIIEFSQKNLYIRKVTVQLMEELFVFKNNKVLFDGQEIPEFHFSGHAQVYWVSSMFVQVHTTIGINFQIQMSPEIHLFIDAPDPSSDKIKGLCGNSNSDTTDDFTTNSGIIENSAKPFAMSWSLLNCFGNIPTTCTNLENENYAHEKCAVLNQPTGIFAKCHPHIPTDYYYTACIQRICNSAGSRRQGLCIGLASYAKACAGVGVVIGDWRRITGCDLKCQKNQEFSYSMHTCNRTCNSLTGHDIRCGMNDDAVEGCGCPEGTHLNQGQTCCPKEECGCIYYGGIAAPGPVVIAGQKCDCKNGILNCLPNCDCRNGKVCVSCSEGQHKRVQKTCDYISKPKGTRENCKSGCYCPDHQYEDHHGNCVSLDDCTCVFSGKAFKAGQQVTSNCKTCTCYRGQWHCIEKPCPGQCQVYGNGHYQTFDSKWFRFSGQCLYTLVQDSCDMRRGTFSIRVESVPCCEEVLTCSRNIILDLKGQVTLTLRDMQVTRRLHEGWTGQDDSLYSIHTLGLYQHAL

SEQ ID NO: 4 - DNA, 2197 bp, Gasterosteus aculeatus
gcagcatgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgtccaggtccacacgaccattggtataaacttccaaattcagatgtcccctgaaatccatctgttcatcgacgcacctgacccctccagtgacaagattaaaggtctttgtggcaatagcaacagtgacaccacagatgacttcaccaccaacagcgggatcattgagaactcagctaaaccatttgctatgtcctggagtttgcttaattgtttcggtaacatacccaccacctgcaccaacctggagaatgagaattatgctcatgaaaagtgtgcagtgttaaaccaaccaactgggatatttgctaagtgccaccctcatatcccaactgattactactacacggcttgcatccaaagaatatgtaactctgccggaagtcggagacagggcttgtgtattggtctggccagctacgccaaagcctgcgccggtgttggtgttgtaattggtgactggcttgagcatcaaggtaggccataatctgtgtgtgcatgcacagaacaattgctgtcaggagaagacctcggagacaaaaacgctctccgcgatatgctaccaccctataaccaacagaagagaggaaaaaacgttcacctacaagcacatcacctcttgtgagtgcagactgtgtaacacacaacactgaaaaaatgttgttagcgcagccattttcatcctgttcccttcagatttctaaatcaaatcaaaatatataggactcaaattgtttttagtgattcattttatttcttctgcaaagaaattttctgatctgcttttagtcaaataaaagtaatcattgtctactcaccaaaaaaaaaaaaaaaaaa

SEQ ID NO: 5 - DNA, 1842 bp, Gasterosteus aculeatus
atgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgtccaggtccacacgaccattggtataaacttccaaattcagatgtcccctgaaatccatctgttcatcgacgcacctgacccctccagtgacaagattaaaggtctttgtggcaatagcaacagtgacaccacagatgacttcaccaccaacagcgggatcattgagaactcagctaaaccatttgctatgtcctggagtttgcttaattgtttcggtaacatacccaccacctgcaccaacctggagaatgagaattatgctcatgaaaagtgtgcagtgttaaaccaaccaactgggatatttgctaagtgccaccctcatatcccaactgattactactacacggcttgcatccaaagaatatgtaactctgccggaagtcggagacagggcttgtgtattggtctggccagctacgccaaagcctgcgccggtgttggtgttgtaattggtgactggcttgagcatcaaggtaggccataa

SEQ ID NO: 6 - protein, 613 aa, Gasterosteus aculeatus
MTTQRWILAFCLSLASVFGTVELFKTKEIQTYTCRTFGSGIVQPFKGESYYVRSDCPFKLTSFNVNRGEYSVTIRRGHNGLLVQVEIIINKVTTLLQNGHILVQNKSVSLPYDHTYQHIFKYGIYTRLRSSLLPFTVTWHNVHGGINSLWVTLESELCTDMCGLCGKQNVAGHRDELIRDSKLHDHRCKIRDPVLRKNHICRRFFLKTKNCLQDNNSHYHRLCKENICGFENSQSIFCPFFQEVASQCNQSRINRFWRRLTKCAKPRCPGDLIYEKKGPAFIPSCSNPNPAPFYQELTETCSCPEGKVLNNCEKGYRCIPKSSCSCEFAGKTYGNGEIRSSRCQSCTCDGGKWRCSENFCHRRCVIEGQFVTTFDGKQYVLPNKCLYVASKGPNWIIIIEFSQKNLYIRKVTVQLMEELFVFKNNKVLFDGQEIPEFHFSGHAQVYWVSSMFVQVHTTIGINFQIQMSPEIHLFIDAPDPSSDKIKGLCGNSNSDTTDDFTTNSGIIENSAKPFAMSWSLLNCFGNIPTTCTNLENENYAHEKCAVLNQPTGIFAKCHPHIPTDYYYTACIQRICNSAGSRRQGLCIGLASYAKACAGVGVVIGDWLEHQGRP

SEQ ID NO: 7 - protein, 472 aa, Gasterosteus aculeatus
MTTQRWILAFCLSLASVFGTVELFKTKEIQTYTCRTFGSGIVQPFKGESYYVRSDCPFKLTSFNVNRGEYSVTIRRGHNGLLVQVEIIINKVTTLLQNGHILVQNKSVSLPYDHTYQHIFKYGIYTRLRSSLLPFTVTWHNVHGGINSLWVTLESELCTDMCGLCGKQNVAGHRDELIRDSKLHDHRCKIRDPVLRKNHICRRFFLKTKNCLQDNNSHYHRLCKENICGFENSQSIFCPFFQEVASQCNQSRINRFWRRLTKCAKPRCPGDLIYEKKGPAFIPSCSNPNPAPFYQELTETCSCPEGKVLNNCEKGYRCIPKSSCSCEFAGKTYGNGEIRSSRCQSCTCDGGKWRCSENFCHRRCVIEGQFVTTFDGKQYVLPNKCLYVASKGPNWIIIIEFSQKNLYIRKVTVQLMEELFVFKNNKVLFDGQEIPEFHFSGHAQVYWVSSMFVQVHTTIGINFQIQGQHRRP

SEQ ID NO: 8 - DNA, 1419 bp, Gasterosteus aculeatus
atgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgttcaggtccacacgaccattggtataaacttccaaattcaggggcagcatcgtcggccgtga

SEQ ID NO: 9 - DNA, 1457 bp, Gasterosteus aculeatus
catcaggcagcatgacaacccagcgatggattctggcattctgcctttcactggcctcagtttttggcacagtggaattgttcaagaccaaagaaattcagacttacacttgcaggacatttggcagcgggatcgtccagccttttaagggggagagttactatgttcggtccgactgcccgttcaaactcacaagcttcaacgtcaaccggggggaatattctgtcaccatacggcgaggtcacaacgggctgttggtccaagtcgagatcatcatcaacaaagtcacaacacttttgcagaatggccacatcctagtgcagaacaaaagtgtttcacttccatacgaccacacctaccagcatatctttaaatacggcatctacactagactgaggagctcgctgcttccttttactgtcacctggcacaatgtacatgggggaataaactctctgtgggtgacactggagtcggagctgtgcaccgacatgtgtggactgtgtggaaaacaaaacgttgcaggccacagggacgagttgatcagagacagcaagcttcatgaccacagatgtaaaatcagagatcctgtgttgcgaaaaaatcacatatgccgtcgattctttctgaaaaccaagaactgtctgcaagacaacaattctcactatcaccgactctgtaaagagaacatttgtggctttgaaaacagccagagcatcttctgtcctttcttccaagaagttgcaagccagtgtaaccaatcaagaatcaaccgattttggagacgtttaaccaaatgtgcgaagccgaggtgtccaggagacctgatttatgagaaaaaaggtccagcatttattcccagctgctccaacccgaaccctgcacccttctaccaggaactcactgaaacctgttcctgtccagagggtaaagttttgaataattgtgaaaagggctatcgctgtatacctaaatccagctgctcttgtgagtttgctggcaagacctacggaaacggagaaatacggagttccaggtgtcagtcatgtacgtgtgatggtgggaaatggcgatgctcagagaacttttgccacagaagatgtgtcattgaaggccagtttgtgacaacattcgatggaaaacaatatgtcctccctaacaaatgtttatatgtggcttcaaagggtcccaactggataataataatagagttttcgcaaaaaaacctctacattagaaaggttacggttcagctcatggaggaactgtttgtattcaaaaacaacaaggttttgtttgatggacaggagatccctgaattccatttttctggtcatgctcaggtttactgggtgtcctccatgttcgttcaggtccacacgaccattggtataaacttccaaattcaggggcagcatcgtcggccgtgataacccagaaacaccgaaactacaaca

(19) United States
(12) Patent Application Publication
(10) Pub. No.: US 2004/0072183 A1
(43) Pub. Date: Apr. 15, 2004

(54) Adhesive protein and uses thereof

(76) Inventors: Per-Erik Olsson, Orebro (SE); Jana Jaas, Orebro (SE); Iwan Jones, New York, NY (US); Staffan Jakobsson, Akersberga (SE); Bertil Borg, Taby (SE)

Correspondence Address: Michael R Ward, Morrison & Foerster, 425 Market Street, San Francisco, CA, 94105-2482, US

(21) Appl. No.: 10/363,485

(22) PCT Filed: Aug. 31, 2001
(86) PCT No.: PCT/SE01/01853

(30) Foreign Application Priority Data
Sep. 1, 2000 (SE) 0003099-9

(51) Int. Cl. C12Q 1/68; C07H 21/04; C07K 14/46

(52) U.S. Cl. 435/6; 435/69.1; 435/320.1; 435/325; 530/350; 530/395; 536/23.5

(57) ABSTRACT

An adhesive protein, present inter alia in the stickleback nest building glue has now been isolated and purified. Its amino acid sequence and corresponding coding polynucleotide sequences are isolated and sequenced. The protein finds both medical and technical uses, whereas both the protein and the polynucleotide sequence can be used in analyses, determining the presence and influence of androgenic substances, for example androgenic pollutants, present in the aquatic environment.

### FIELD OF THE INVENTION

This invention concerns an adhesive protein, isolated and purified by the present inventors, and its uses in analytical, medical and technical applications. Further, the nucleotide sequence encoding said protein and its amino acid sequence are disclosed, including uses thereof.

### BACKGROUND OF THE INVENTION

Analytical Applications:

The increasing pollution of the environment is one consequence of our highly developed technical society. It has quite recently been discovered, that certain organic compounds, both pharmaceuticals and industrial chemicals, such as softening additives in plastics, are hormonally active or hormone mimicking in living organisms. Unmetabolized etinyle oestradiol from oral contraceptives has been detected in municipal effluents. Pulp mills are another possible source of hormonally active compounds, such as phytoestrogens. There are now alarming indications that these compounds interfere with both early embryo and larval development as well as sexual differentiation in water living animals. Besides estrogenic responses, masculinization of female fish has been observed.

Present models for detecting these compounds and monitoring their effects include observations (biopsies) of fish and frogs in the area under investigation, experiments involving laboratory animals etc. Hitherto, cDNA probes have been developed for the oestrogen receptor (ER), vitellogenin (VTG) and vitelline envelope proteins (VEP).

There remains however an urgent need for the development of new, more sensitive markers for hormonal responses such as androgenic responses.

Medical Applications:

Bio-degradable gels, adhesives and, in some applications, binders and fillers, are needed in modern health care. Wound healing is disturbed by the presence of necrotic tissue. Severe burns, severe ulcers, deep cuts etc where necrotic tissue is present and difficult to remove, are today treated with waterbased gels, softening and removing necrotic tissue. One gel, presently on the market contains water, sodium carboxymethyl cellulose, and calcium alginate.

Alginates are hydrocolloids obtained from seaweed. They are well tolerated by the body and biologically degradable. Alginates are used in health care as components in wound dressings, fillers for deep and/or exudating wounds, compresses etc. Alginate products are usually delivered in the forms of pads, ropes or ribbons. When absorbing the exudate, the alginate products form a gel which maintains a moist environment and protects the wound.

Another biologic polymer used in health care is collagen, which plays an important part in wound healing. Collagen can be extracted from animal sources and used as a matrix for wound healing, skin reconstruction etc. Artificial skin grafts, used in dermatoplastic surgery and reconstructive surgery, are presently based on animal skin, which has been subjected to chemical treatments, leaving only the supporting collagen matrix structure, which will be colonised by the patients own tissue.

Biodegradable adhesives are used for closing of minor wounds and incisions, for example in facial wounds and plastic surgery. Likewise, biodegradable sutures are currently in use.

Technical Applications:

The development of a glue which would be both possible to apply and to cure in moist environments or even under water, is highly desired. Presently used glues and sealing agents are either epoxy based, cement based or based on synthetic polymers. Both the epoxy compounds and the synthetic polymers may leach and constitute a risk to the environment. Their application often require mechanical working or kneading of the glue or sealing agent, in order to remove the water present on the surfaces. There is a need for new glues or sealing agents, better adapted for use in moist environments or for underwater use and more environmentally friendly than the present products.

### PRIOR ART

It has been a well-known fact that the male three-spined stickleback (Gasterosteus aculeatus) uses some kind of adhesive substance to build its typical “nest” of plant material. In Fish Physiology and Biochemistry 20: 79-85, 1999, Jakobsson et al. disclose the stickleback nest building glue as being characterised and found to consist almost entirely of one major protein with an apparent molecular size of 203 kDa as determined from urinary bladder content. The protein was named “spiggin” after the Swedish name for stickleback, spigg. Spiggin production was shown to be inducible by 11-KT treatment and said to be the hitherto only known protein to be induced by 11-KT.

### SUMMARY OF THE INVENTION

The present inventors now make available a characterised and purified biopolymer with many uses in analysis, medicine, and other technical applications as disclosed in the following description, examples and claims.

Compared to what was known prior to the priority date of the present application, the inventors have shown that what was thought to be one single protein, “spiggin”, in fact is a multimerised protein complex comprising at least three isoforms. It is in a way unfortunate, that the previously disclosed aggregate and the surprisingly identified and characterised variants still carry the same name, as this may be the source of confusion. The present inventors has however chosen to adhere to the name “spiggin” although it in the following description and claims is used to encompass the protein complex, as well as single isoforms, as determined by the context.

The present inventors have isolated and sequenced the nucleotide sequence, resulting in a full-length sequence, comprising approximately 4.2 kb (SEQ. ID. NO. 1) and two shorter sequences, one approximately 2.2 kb sequence (SEQ. ID. NO. 4) and one approximately 1.4 kb sequence (SEQ. ID. NO. 9). Further, the corresponding coding sequences have been identified and sequenced (SEQ. ID. NO. 2, SEQ. ID. NO. 5, and SEQ. ID. NO. 8). Finally, the inventors have isolated and purified the corresponding proteins (SEQ. ID. NO. 3; SEQ. ID. NO. 6, and SEQ. ID. NO. 7). The sequences are attached in the form of a sequence listing, prepared using PatentIn 2. 1. The sequences will be referred to by their sequence identity numbers (SEQ. ID. NO.) and are incorporated in the description and claims in their entirety.

### SHORT DESCRIPTION OF THE DRAWINGS

The invention will be described in closer detail below, with reference to the examples and figures, in which

FIG. 1 shows a Northern blot with the molecular mass of the cDNA splicing variants of spiggin (Lane 1: RNA Ladder, Gibco BRL; Lane 2: Control kidney total RNA; Lane 3: 11-KT treated kidney total RNA);

FIG. 2 shows the result of a slot blot analysis of tissue specific spiggin expression within selected tissues of male three-spined stickleback (Lane 1: fish 1; Lane 2: fish 2; Lane 3: fish 3);

FIG. 3 shows the 11-KT inducibility of the gene product in the form of a dose response analysis (% dpM/=m²) using the following substances: 11-KT 4, 11-KT 20, 11-KT 100, 11-KT 500, 11-KT 2500, cortisol 100, cortisol 500, cortisol 2500, oestrogen 2500, testosterone 2500, progesterone 2500, 5-α DHT 2500, and control (μg/100 μl coconut oil);

FIG. 4A shows the Coomassie brilliant blue staining of kidney and urinary bladder content on a polyacrylamid gel (SDS-PAGE, Lane 1: 11-KT treated kidney extract; Lane 2: 11-KT treated bladder extract);

FIG. 4B shows the cross-reactivity of proteins from kidney and urinary bladder with a specific spiggin polyclonal antibody (Western analysis employing spiggin KTK 16 antibody, Lane 1: 11-KT treated kidney extract; Lane 2: 11-KT treated bladder extract);

FIG. 5 shows the methyltestosterone (MT) induction of spiggin mRNA and inhibition with 1000×flutamide; and

FIG. 6 shows the spiggin mRNA levels in a Swedish pulp mill location, proving the applicability of the inventive concept in environmental analysis.

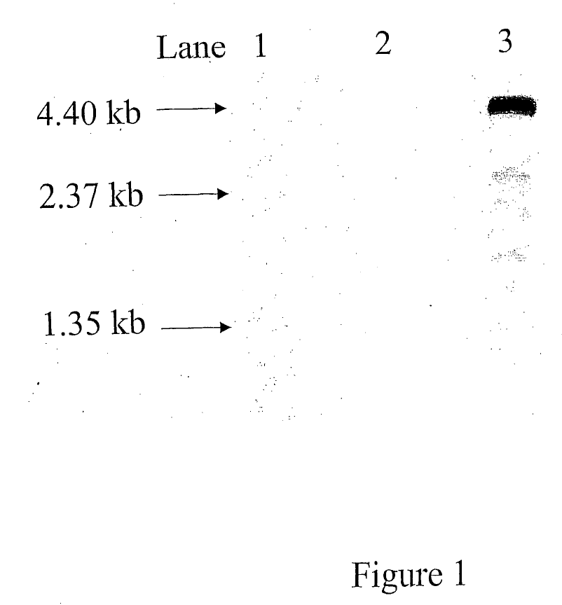

### DESCRIPTION OF THE INVENTION

The present invention makes available a substantially pure adhesive protein, comprising the amino acid sequence of SEQ. ID NO. 3 or fragments thereof, such as the shorter sequences of SEQ. ID. NO. 6 and SEQ. ID. NO. 7, including functionally equivalent fragments or variants thereof. The term “functionally equivalent” is meant to encompass proteins, or polynucleotide sequences exhibiting equivalent properties with respect to any desired quality in question, such as the adhesive property, medical properties, the capability to function as an indicator etc.

The sequence data surprisingly reveals that the protein has a unique structure and organisation with autocatalytic sites and binding sites for phosphorylation, carbohydrates, and lipids. Identification of the protein in the kidney, where it is synthesised, demonstrates that it consists of a 130 kDa protein with smaller splicing variants (as confirmed by nucleotide sequencing).

This also demonstrates that the previous identification of a 203 kDa protein in fact was a multimer complex of at least three here identified and characterised proteins. Interestingly, these proteins show only low homology to other known proteins, with the highest similarity being 28% to mucins. Post-translational modifications, by glycosylation, account for a minor increase of the molecular mass of the protein.

The invention also makes available the DNA and cDNA sequences, enclosed as SEQ. ID. NO. 1, 4, and 9; and 2, 5, and 8, respectively. The invention also encompasses homologous sequences, and in particular sequences exhibiting a homology greater than 50%, preferably greater than 70% and most preferably greater than 90%.

By operatively inserting the coding sequence in a suitable vector, and transfecting a host cell with said vector, the host cell can be made to produce larger quantities of the protein in vitro. E. coli is one suitable host cell. It will however fall within the abilities of a skilled person to select and apply a suitable vector and host cell culture. Thus, having access to the sequence data disclosed herein, the large scale production of the protein becomes a question of routine work.

Analytical Applications:

According to the present invention, the protein and/or information derived thereof, such as the sequence data, is used for the determination of hormone disrupting or androgenic compounds in the environment.

Methods:

One embodiment of the invention is a method for the detection of androgenic compounds in biological systems comprising the use of specific antibodies, for use in different protein detection systems. In an ongoing study, the usefulness of spiggin antibodies to detect androgenic effects in pulp mill effluent waters is investigated. Preliminary results confirm that spiggin is a good androgenic biomarker.

Another embodiment is a method for the detection of androgenic compounds in biological systems comprising the use of the spiggin cDNA in mRNA detection systems. In an ongoing study, the usefulness of spiggin mRNA to detect androgenic effects in pulp mill effluent waters is investigated. Preliminary results confirm that spiggin is indeed a good androgenic biomarker.

Furthermore, specific peptide sequences are to be used as standards in antibody detection systems to allow proper quantification of spiggin. According to one embodiment of the invention, the peptides used for production of the antibodies are used as control standards in the detection of androgenic compounds.

Western Blot:

One embodiment of the invention is a Western blot method to detect spiggin protein comprising the following steps:

1) collection of a kidney or urinary bladder sample from stickleback and mixing it with gel loading buffer,

2) gel electrophoresis of the protein on SDS-polyacylamid gels,

3) transfer of the protein to nylon or nitro-cellulose membrane,

4) blocking of membrane using milk powder or equivalent solutions,

5) incubation with primary antibody directed against native spiggin, synthetic spiggin peptide, or recombinant protein,

6) washing of membrane,

7) incubation of membrane with secondary antibody,

8) washing of membrane, and

9) detection of specific signal using a selection of optional detection systems.

ELISA:

Another embodiment of the invention is an enzyme linked immunosorbent assay (ELISA) comprising the following steps:

1) collection of kidney or urinary bladder sample from stickleback,

2) dilution of samples in coating buffer,

3) dilution of positive control consisting of native spiggin, synthetic peptide or recombinant protein in coating buffer,

4) addition of samples to three wells each,

5) addition of positive control to three wells each to create a standard curve,

6) washing of the wells 3 times with washing buffer,

7) addition of blocking/dilution buffer to each well,

8) incubation at room temperature for required time,

9) washing of wells 3 times with washing buffer,

10) dilution of primary antibodies (against native spiggin, synthetic peptide or recombinant protein) in blocking/dilution buffer and addition to each well,

11) incubation at an appropriate temperature for required time,

12) washing of the wells 3 times with washing buffer,

13) dilution of secondary antibody in blocking/dilution buffer and addition to each well,

14) incubation at appropriate temperature for the required time,

15) washing of the wells 3 times with washing buffer,

16) addition of substrate solution to each well,

17) incubation at room temperature for the required time,

18) stopping of reaction, and

19) measurement of signal using an optional detection method.

Kits for the detection of androgenic compounds:

Another embodiment of the invention is a kit for the detection of androgenic compounds, comprising at least antibodies raised against one or several synthetic peptides designed against antigenic epitopes of the protein, synthetic protein or recombinant protein and the specific peptide or peptides as standard.

According to a further embodiment, the necessary components for ELISA (enzyme linked immunosorbent assay) detection are also included in such a kit. An ELISA Kit according to the invention would preferably comprise the following:

1) polyclonal or monoclonal antibodies raised against native spiggin,

2) synthetic peptides or recombinant protein designed against antigenic epitopes of the protein,

3) the specific synthetic peptide or recombinant protein as standard,

4) ELISA coating plates,

5) coating buffer,

6) phosphate buffered saline,

7) washing buffer

8) blocking buffer

9) secondary antibody directed against rabbit for polyclonal primary antibody or mouse for monoclonal primary antibodies,

10) detection substrate such as horseradish peroxidase substrate or other equivalent substrate,

11) specification chart for the primary and secondary antibodies and the peptide standard, and

12) a manual containing instructions for use and requirements of additional components.

A kit according to the present invention may also comprise only part of the above listed components, the necessary components being antibodies or standards, produced using the inventive protein and/or information derived thereof, such as the sequence data disclosed herein or homologous sequences, including functionally equivalent sequences.

Medical Applications:

According to further embodiments of the invention, the adhesive protein or protein complex is used in medical applications, for example as a component in wound dressings and bandages, in particular in such applications where the biodegradable properties of the protein are needed.

Due to spiggin being a viscous adhesive protein that functions in water and other moist environments it could be used e.g. for antibiotic therapy and coating of dressings. By mixing the protein with selected pharmaceutical compounds, a slow release composition is achieved. The adhesive properties also aid in immobilising the pharmaceutical to a specific location of the skin or in the body of a patient, undergoing treatment.

Since the protein has an ability to attach to surfaces, and to form an attachment between surfaces, it may be used as a tissue adhesive. The capability of attaching paper and latex samples to human skin shows that spiggin also may be used as an adhesive for plasters, adhesives, bandages, patches and dressings etc. The protein may also be useful in orthopaedics as a glue to keep or hold joint replacements together. Among other uses of this protein is the possible use as a biomechanical shock absorber due to its viscous nature. Such biomechanical components include artificial cartilage, intervertebral discs and/or parts thereof.

Technical Applications:

There is at present no glue/paste or cement that works well under water. Since the protein has an ability to attach to surfaces, and to connect surfaces, as tested by attaching small pieces of wood to each other in a wet environment, it may be used as an underwater adhesive/glue. In addition to being used as an underwater glue or adhesive, the protein may also be used as an underwater sealant.

One embodiment of the present invention is the application of the adhesive protein as such, derivatives thereof or information derived thereof for the production of a glue or an adhesive for use in moist environments. Moist environments in this context include both aquatic environments, objects and surfaces in contact with water; sea water, fresh water, high humidity, steam and/or condensation. The applications can be found in both natural or man-made environments and even on or within an animal or human body.

### EXAMPLES

### Materials and Methods

1. Protein Sequencing.

Amino acid analysis was performed on the 203 kDa region from gene electrophoresis by ion-exchange chromatography as described by Jakobsson et al., Fish Physiol Biochem 20: 79-85 (1999). One potential N-terminal and several potential internal sequences were obtained. These were Spiggin 1 (KTKEIQTY), Spiggin 2 (KAVLSIHPDFSVVK), Spiggin 3 (KENYISHK), Spiggin 4, (SYYVR) Spiggin 5 (RGTFSIR), Spiggin 6 (LYIRK), Spiggin 7 (IRDPVLRK) and Spiggin 8 (TAYSWV). Of these, sequence 1, 4, 5 and 6 were confirmed to be spiggin following cloning of the cDNA.

2. Hormone Treatment.

Hormones at different concentrations were dissolved in 100 ml of coconut oil and placed within silastic capsules (Dow Corning, internal diameter 0.6 mm, outer diameter 1.2 mm, length 5 mm). The sealed capsules were implanted into the abdominal cavity of each fish which were subsequently placed in aquaria containing 200 l of brackish water (0.5% salinity) at 17° C. under a stimulatory photoperiod of L:D 16:8. Following one month exposure the fish were sacrificed and the required organs removed by dissection and stored at −70° C.

3. RNA Extraction.

Total RNA was extracted from all tissues using TRI reagent” (Sigma). The mRNA fraction was isolated using the Poly(A) Quik′ mRNA purification kit (Stratagene). All preparations were stored at −70° C.

4. Reverse Transcriptase—Polymerase Chain Reaction (RT-PCR).

Complementary DNA was synthesised from 0.5 mg of total RNA using the First Strand cDNA synthesis kit (Amersham Pharmacia Biotech). PCR was performed with 500 ng of cDNA termplate in a final volume of 50 ml. The reaction mix contained 0.2 mM dNTPs, 5 ml 10×reaction buffer, 2.5 nM of magnesium chloride, 1.25 U of Taq DNA polymerase and 25 pmol of Spiggin 1 (5′-CARACIAARGARATICARAC-3′) and Spiggin 3 (5′-TTRTGIGAIATRTARTTYTCYTT-3′) oligonucleotides. Following an initial denaturation step at 95° C. for 3 min, amplification was performed at 95° C. for 1 min, 50° C. for 1 min, 72° C. for 1 min for 40 cycles. PCR products of approximately 600 bp were generated which were visualised by agarose gel electrophoresis according to Sambrook et al., Molecular Cloning: A laboratory manual, 2^{nd} Ed. (1989).

5. Sequencing of RT-PCR Products.

All generated RT-PCR products were ligated into pGEM-T′ (Promega) and transformed in competent E. coli cells according to Sambrook et al., (1989). Positive colonies were grown under antibiotic selection over night and recombinant plasmids were isolated using the Wizard′ Plus SV miniprep system (Promega). Cycle sequencing was performed using the Thermo Sequenase v2.0 sequencing kit (Amersham Pharmacia Biotech). The reactions were resolved on an ABI Prism″ 377 DNA sequencer (Perkin Elmer) and the data obtained analysed using EditView v1.0.1 (Perkin Elmer).

6. Tissue Distribution and Dose Response Analysis.

5 mg total RNA was combined with 90 ml of 20×SSC and 60 ml 37% formaldehyde. The final volume was adjusted to 300 ml with MQ water. Each preparation was transferred onto nylon membrane (Amersham Pharmacia Biotech) using a Minifold II Slot Blot apparatus (Schleicher & Schuell). The RNA was bound onto the membrane by cross-linking in a CL-1000 UV apparatus (UVP). Membranes were radioactively probed as described below (Paragraph 10) and the relative levels of mRNA were determined using a GS-250 Molecular Phosphoimager coupled to the Molecular Analyst (Version 1:41) package (Bio Rad). All dose-response determinations were performed in triplicate and the results were expressed as the mean (±SD) for each hormone and dose assayed.

7. Northern Analysis.

Northern analysis of total RNA was performed according to Sambrook et al., (1989) supra. Membranes were radioactively probed and visualised as described below (Paragraph 10).

8. Slot Blot Analysis

Total RNA was extracted from mature male tissues, from three individual fish, using TRI reagent® (Sigma). Aliquots of 5 mg total RNA were mixed with denaturing solution (6×SSC, 7% (v/v) formaldehyde) and transferred onto nylon membrane (Amersham Pharmacia Biotech) using a Minifold II Slot Blot Apparatus (Schleicher and Schuell). Membranes were probed using a randomly primed [a32P]-dCTP radiolabelled spiggin cDNA fragment (636 bp) that was isolated by RT-PCR and sequenced as above. Hybridisations were performed at 65° C. O/N (6×SSC, 0.1% (w/v) SDS, 100 mg ml-1 tRNA and 5×Derhardt's solution). The membranes were washed for 2×30 min periods at 42° C. and 65° C. in 0.1×SSC/0.1% (w/v) SDS and exposed to Hyperfilm®-MP film (Amersham Pharmacia Biotech) at −70° C. The films were visualised using a Curix 60 Filmn Developer (AGFA).

9. cDNA Library Construction and Screening.

A cDNA library was generated from the poly-A fraction of stickleback kidney RNA which was isolated as described previously (Paragraph 3). The library was constructed in Lambda ZAP Express′ (Stratagene). Screening was performed according to Sambrook et al., (1989) supra, using radioactive probing as described below (Paragraph 10). Several individual clones were identified following four rounds. These were isolated according to the Lambda ZAP Express′ instruction manual and sequenced as described below (Paragraph 9).

10. Sequencing of Library Isolated Clones.

Initial sequencing was done as described in Paragraph 5 to confirm the identity of individual spiggin cDNA clones. The selected clones were thereafter sequenced by Cybergene AB, Huddinge, Sweden (100% Patent Sequencing).

11. Radioactive Probing.

All membranes were probed using a 600 bp [a32P]-dCTP radiolabelled cDNA fragment of spiggin which was previously isolated by RT-PCR (Paragraph 4). Hybridisation was performed at 65° C. O/N in 6×SSC, 0.1% (w/v) SDS, 100 mg ml-1 tRNA and 5×Denhardt's solution (0.1% (w/v) BSA, 0.1% (w/v) ficoll, 0.1% (w/v) polyvinylpyrrolidone). The membranes were washed for 2×30 min periods at 42° C. and 65° C. in 1×SSC, 0.1% (w/v) SDS and exposed to Hyperfilm″-MP film (Amersham Pharmacia Biotech) at −70° C. O/N. The films were visualised using a Curix 60 film developer (AGFA).

12. Production of Polyclonal Antibodies.

Polyclonal antibodies were generated by Agri Sera AB. The following peptides were employed: Spiggin KTK 16 (KTIEIQTYTSRTFGS-C) and Spiggin HRD 16 (HIRDELIRDSHLHDHR-C) which correspond to the N-terminal sequence and amino acid sequences at positions 149-164, respectively of the spiggin protein.

13. SDS-PAGE.

Protein samples were resolved by SDS-PAGE at a constant current of 50 mA for 1 h according to Laemnmli (1970) using 10% and 5% Tris-glycine running and stacking gels, respectively. Proteins were subsequently visualised by staining with Coomasie brilliant blue, or silver nitrate according to Sambrook et al., (1989) supra.

14. Western Blot Analysis.

Proteins were transferred onto nitro-cellulose membrane (Amersham Pharmacia Biotech) following SDS-PAGE using the Trans-Blot′ Semi Dry Electrophoretic Transfer Cell (Bio Rad) at 15 V for 30 min in a buffer which consisted of 48 mM Tris, 39 mM glycine and 20% methanol (pH 9.2). Immunodetection was performed according to Sambrook et al., (1989) using sera raised against either Spiggin KTK 16 or Spiggin HRD 16 as primary antibodies (Paragraph 11) and rabbit immunoglobulin HRP conjugate (DAKO) as secondary antibody, respectively. Proteins were visualised using the HRP substrate kit (Bio Rad).

15. Adhesive Properties.

Spiggin protein obtained from urinary bladders was tested for its ability to hold together biological material such as pieces of wood. This ability was tested by holding together the pieces and then determining if they would remain attached. The ability to adhere to human skin was tested according to the following protocol:

The male stickleback has a high content of spiggin in its urinary bladder during the breeding season. Three male stickleback were collected and their urinary bladder content extracted. This urinary bladder content will below be called “spiggin”. Spiggin was compared to a solution of bovine serum albumin (BSA), mineral oil, and water for its ability to attach 1 cm² samples of KIM® wipe paper or latex (cut from a surgical glove) to human skin. The site of attachment was the lower side of the forearm of the testperson. The experiment was performed thrice. An initial test was performed using only spiggin and water and the samples observed for 60 minutes (Experiment I). This was followed by two experiments where the samples were observed for 20 minutes (Experiments II and III). At the end of each experiment, the samples were removed from the skin using a fine forceps. Attachment was determined as none (−), very weak (+/−), weak (+), strong (++), and very strong (+++). If a sample detached on its own, the time to detachment was registered.

16. Methyltestosterone Induction of Spiggin mRNA

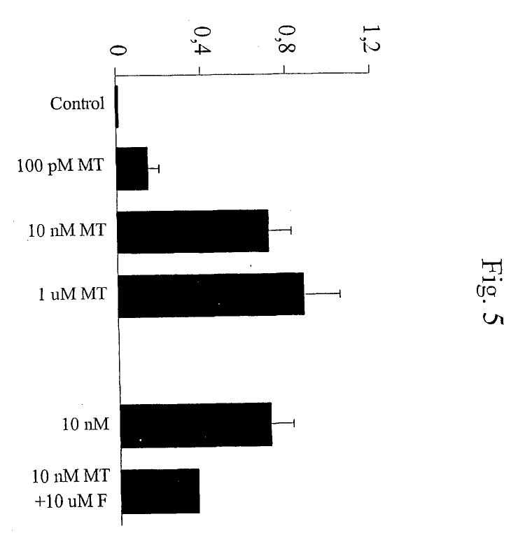

Juvenile three spined sticklebacks, with a body weight of approximately 100 mg, were exposed to different doses (100 pM, 10 nM and 1 uM) of methyltestosterone or to a combination of 10 nM methyltestosterone and 10 uM flutamide (an antiandrogen). After 10 days the fish were sacrificed. Whole fish were homogenized and spiggin mRNA levels were determined using slot blot analysis. The results are presented in FIG. 5.

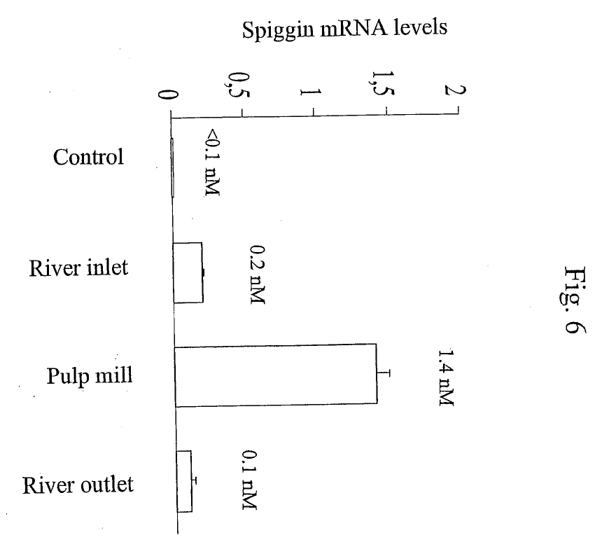

In a separate experiment juvenile three spined stickleback were exposed to water samples collected from a Swedish river and a pulp mill in this river. The fish were exposed to 10×diluted pulpmill and river outlet water and undiluted incoming river water for 10 days. As a control a separate group was exposed to undiluted spring water. Whole fish were analysed for spiggin mRNA content using slot blot analysis. The methyltestosterone exposure was plotted to create a standard curve. The spiggin levels in the water exposed fish were compared to the standard curve and presented as nM methyltestosterone equivalents. The results are presented in FIG. 6.

Results

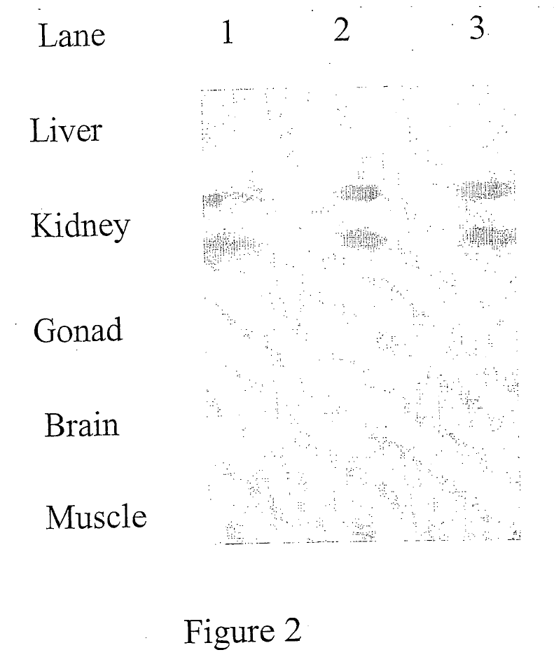

Sequencing of the stickleback kidney cDNA library revealed the presence of at least three splicing variants of the spiggin protein. These variants are encoded for by a 4.2 kb (SEQ ID NO. 1), a 2.2 kb (SEQ ID NO. 4), and a 1.6 kb (SEQ ID NO. 8) mRNA. Northern blot analysis of 11KT induced stickleback kidney (FIG. 2) indicates that there are at least three different splicing variants, with the largest being the 4.2 kb mRNA that has been cloned and the 1.6 kb mRNA being the smallest.

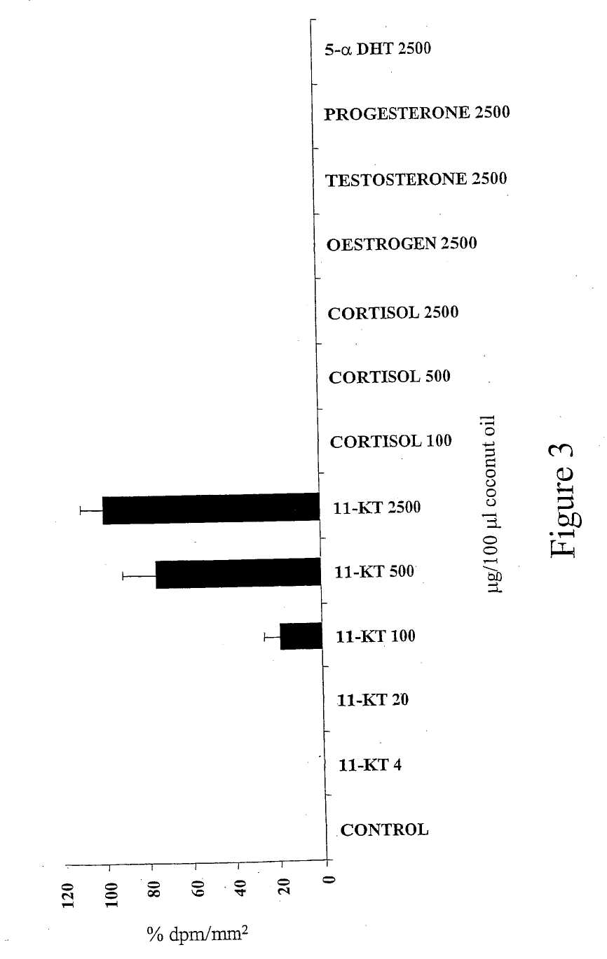

The stickleback spiggin glue protein is exclusively expressed in the kidney of sexually maturing male stickleback (FIG. 2) and is only inducible by 11-ketotestosterone (FIG. 3).

The largest cloned cDNA codes for a 910 amino acid protein with a molecular mass of 103 IDa, which surprisingly was found to be approximately 50% of the previously determined size of spiggin in urinary bladder. The spiggin protein has been estimated to 203 kDa in urine and has been shown to have some glycosylation resulting in a few percent reduction in molecular mass.

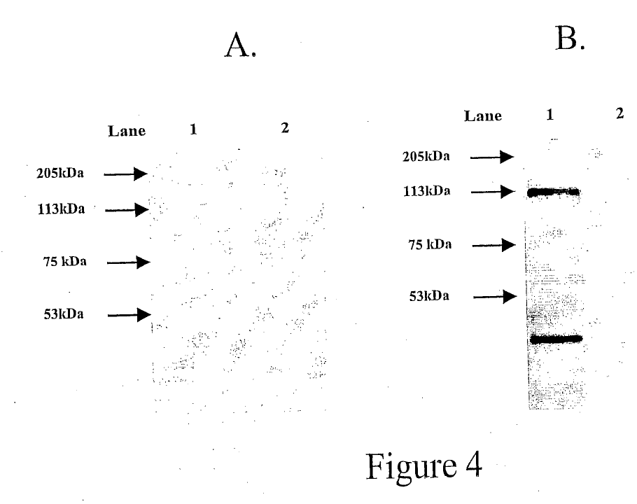

To determine the reason for the discrepancy in molecular mass the present inventors produced 2 polyclonal antibodies. One was directed against the N-terminal region KTKIEQTYTSRTFGS (amino acid 25-39) following the leader sequence, and the other was directed against an antigenic epitope located between amino acid 173 and 188, HRDELIRDSHLHDHRC. Using both these antibodies to detect spiggin in kidney and urinary bladder demonstrated that the antibodies detected the 203 kDa urinary product while this product was not present in kidney (FIG. 4b).

The largest protein detected in kidney was approximately 130 kDa, which is in good agreement with the deduced protein encoded by the 4.2 kb mRNA. Several smaller components, probably reflecting both the splicing variants (51 kDa and 90 kDa) and degradation produces were also detected on the Western blots. Detection of spiggin using Coomasie brilliant blue staining also indicate that the 203 kDa protein is not present in kidney but is a modified product, probably a dimer, that appears first in the urinary bladder (FIG. 4a).

Together these results show that the spiggin gene encodes for at least 3 splicing variants and that the largest of these has an unmodified molecular mass of 103 kDa. Furthermore This protein appears as a 203 kDa product in urinary bladder, probably as a result of multimerisation.

The spiggin protein has also been tested for its ability to hold pieces of wood together in a wet environment. The present inventors found that addition of spiggin resulted in attachment of the pieces. This is in line with the function of spiggin in nature where it is used by the male stickleback to glue together biological material to form a nest.

The experiments evaluating the ability of spiggin to attach showed promising results. Spiggin was able to firmly attach a piece of Kim wipe paper in all three tests. Spiggin attached latex had a fluid attachment for about 10 min before drying out and attaching the latex. The results are presented in the table below:

TABLE 1
Ability of spiggin to attach sample to human skin

| | | |
|---|---|---|
| Solution | KIM Wipe | LATEX |

Experiment I

| | | |
|---|---|---|
| Water | + | 10 minutes |
| Spiggin | + + + | + |

Experiment II

| | | |
|---|---|---|
| Water | + | +/− |
| Mineral oil | − | 15 minutes |
| Bovine serum albumin | − | +/− |
| Spiggin | + + + | + |

Experiment III

| | | |
|---|---|---|
| Water | 4 minutes | +/− |
| Mineral oil | 3 minutes | 10 minutes |
| Bovine serum albumin | +/− | +/− |
| Spiggin | + + + | + |

The ability of spiggin to function as an androgenic marker was tested, both using different doses (100 pM, 10 nM and 1 uM) of methyltestosterone, a combination of 10 nM methyltestosterone and 10 uM flutamide (an antiandrogen), and diluted pulpmill outlet water.

In experiments evaluating the ability of spiggin to be upregulated by androgenic compounds via water it was found that methyltestosterone down to 100 pM was effective at inducing spiggin mRNA. Furthermore, the androgen specificity of the system was confirmed by the inhibiting ability of flutamide (an antiandrogen) when added together with to methyltestosterone.

Confirmation that androgenic responses are present in the environment was obtained by testing water from a river and a pulpmill. The river sampling point showed weak androgenic effects comparable to a 200 pM methyltestosterone exposure. This water is down stream of a major sewage treatment plant. Higher levels of spiggin mRNA induction was observed within the pulp mill with levels reaching 1400 pM methyltestosterone equivalents. Finally, the ability of the pulp mill to purify the outlet water was observed by the lower androgenicity of the River outlet water. Together these studies demonstrate that spiggin is a highly sensitive biomarker for androgenic effects. The results are presented in FIGS. 5 and 6.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1. A substantially purified adhesive protein comprising the amino acid sequence of SEQ ID NO: 6 or fragments thereof.

2. A substantially purified adhesive protein comprising the amino acid sequence of SEQ ID NO: 3 or fragments thereof.

3. A substantially purified adhesive protein comprising the amino acid sequence of SEQ ID NO: 7 or fragments thereof.

4. A protein or protein fragment according to any one of claims 1-3, wherein said protein or fragment exhibits a repetitive structure of Cystein rich regions bisected by von Willebrand factor like protein D-domains.

5. An isolated and purified polynucleotide sequence encoding a protein of any one of claims 1-4.

6. A polynucleotide sequence which hybridises under stringent conditions to the sequence of claim 5.

7. An isolated and purified polynucleotide sequence comprising SEQ ID NO: 5 or functionally equivalent variants thereof, said sequence and variants coding an adhesive protein.

8. An isolated and purified polynucleotide sequence comprising SEQ ID NO: 2 or functionally equivalent variants thereof, said sequence and variants coding an adhesive protein.

9. An isolated and purified polynucleotide sequence comprising SEQ ID NO: 8 or functionally equivalent variants thereof, said sequence and variants coding an adhesive protein.

10. An isolated and purified polynucleotide sequence comprising SEQ ID NO: 4 or functionally equivalent variants thereof, said sequence and variants coding an adhesive protein.

11. An isolated and purified polynucleotide sequence comprising SEQ ID NO: 1 or functionally equivalent variants thereof, said sequence and variants coding an adhesive protein.

12. An isolated and purified polynucleotide sequence comprising SEQ ID NO: 9 or functionally equivalent variants thereof, said sequence and variants coding an adhesive protein.

13. A polynucleotide sequence which is complementary to the sequence according to any one of claims 5-12.

14. An expression vector comprising a sequence according to any one of claims 5-12.

15. A host cell containing the vector of claim 14.

16. A method for producing a polypeptide comprising the amino acid sequence of SEQ ID NO: 6 or fragments thereof, the method comprising the steps of:
culturing the host cell of claim 18 under conditions suitable for the expression of the polypeptide; and
recovering the polypeptide from the host cell culture.

17. A purified antibody binding specifically to a protein of any on of claim 1-4.

18. An antibody according to claim 17, characterized in that said antibody is monoclonal.

19. An antibody according to claim 17, characterized in that said antibody is polyclonal.

20. A method for the detection of androgenic compounds in biological systems, characterized in that said method comprises the following steps:
collection of samples of kidney or urinary bladder from sticklebacks,
separation of proteins,
transfer to membrane, and
detection of spiggin using antibodies directed to the protein of claim 1, 2, 3 or 4.

21. A method for the detection of androgenic compounds in biological systems, characterized in that said method comprises the following steps:
collection of samples of kidney or urinary bladder from sticklebacks,
coating of ELISA plates with samples and standard,
blocking and washing wells,
addition of a monoclonal antibody directed to the protein of claim 1, 2, 3 or 4,
incubation and washing,
addition of secondary antibody,
incubation and washing, and
detection of signal.

22. A method for the detection of androgenic compounds in biological systems, characterized in that said method comprises the following steps:
collection of samples of kidney or urinary bladder from sticklebacks,
extraction of RNA,
separation of RNA on agarose gel,
transfer to nylon membrane,
prehybridisation followed by hybridisation with labelled spiggin cDNA or cRNA,
incubation and washing, and
detection of specific signal.

23. A kit for the detection of androgenic compounds, characterized in that said kit comprises an antibody according to any one of claims 17-19.

24. A kit for the detection of androgenic compounds, characterized in that said kit comprises and antibody according to any one of claims 17-19 and a protein standard based on the protein of claim 1, 2, 3 or 4, buffers, secondary antibodies, detection substrate, specification chart for the primary and secondary antibodies and the peptide standard and instructions for use.

25. A therapeutic composition, characterized in that it comprises an effective amount of a protein according to any one of claims 1-4.

26. A therapeutic composition according to claim 25, characterized in that it further comprises a compound selected among antibacterial, antiviral, antifungal, analgesic, moisturising, and dehydrating compounds.

27. A therapeutic composition according to claim 25, characterized in that it further comprises an antibiotic compound.

28. A wound dressing, characterized in that it comprises an effective amount of a protein according to any one of claims 1-4.

29. An implant, characterized in that it comprises a protein according to any one of claims 1-4.

30. Use of a polypeptide according to any one of claims 1-4, for the manufacture of a product for use in or on the body of a mammal.

31. Use of a polypeptide according to any one of claims 1-4, for the manufacture of a medicament.

32. Use according to claim 30, characterized in that the product for use in or on the body of a mammal is chosen among the following products: stents, sutures, wound closure staples, surgical tape, surgical glue, hemostatic fillers and compresses, protective wound covers and/or coatings,

33. An adhesive for application in moist environments, characterized in that it comprises an effective amount of a protein according to any one of claims 1-4 produced in a transgenic host cell.

34. A sealant for application in moist environments, characterized in that it comprises an effective amount of a protein according to any one of claims 1-4 produced in a transgenic host cell.
